(19) United States
(12) Patent Application Publication
(10) Pub. No.: US 2025/0000801 A1
(43) Pub. Date: Jan. 2, 2025

(54) DAROLUTAMIDE PHARMACEUTICAL COMPOSITION, PREPARATION METHOD THEREFOR AND USE THEREOF

(71) Applicant: Sinotherapeutics Inc., Shanghai (CN)

(72) Inventors: Xiaoming QIAN, Shanghai (CN); Yu SHI, Shanghai (CN); Kun LI, Shanghai (CN); Jiansheng WAN, Shanghai (CN); Xiaoxi SHENG, Shanghai (CN)

(21) Appl. No.: 18/712,989

(22) PCT Filed: Nov. 18, 2022
(86) PCT No.: PCT/CN2022/132894

§ 371 Date: May 23, 2024

(30) Foreign Application Priority Data
Nov. 26, 2021 (CN) 202111422274.6

(51) Int. Cl. A61K 9/20; A61K 31/4155

(52) U.S. Cl. CPC A61K 9/2054; A61K 9/2013; A61K 9/2027; A61K 9/2031; A61K 31/4155

(57) ABSTRACT

The present invention relates to a pharmaceutical composition comprising an active ingredient Darolutamide or a pharmaceutically acceptable salt thereof, a carrier material, and a preparation modifier. The present invention also relates to a solid preparation comprising the pharmaceutical composition of the present invention. The present invention further relates to methods for preparing the pharmaceutical composition and the solid preparation of the present invention.

The application claims the priority of Chinese application No. 202111422274.6 filed on Nov. 26, 2021, with the title of “A Darolutamide pharmaceutical composition and preparation process and use thereof”, of which the entire content of the Chinese application is incorporated herein by reference.

### TECHNICAL FIELD

Provided is a Darolutamide pharmaceutical composition and preparation process and use thereof.

### BACKGROUND

Darolutamide has the chemical name of N-[(1S)-2-[3-(3-chloro-4-cyanophenyl)-1H-pyrazol-1-yl]-1-methylethyl]-5-(1-hydroxyethyl)-1H-pyrazole-3-carboxamide, with the following chemical structure.

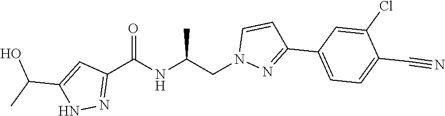 [SMILES: CC(O)c1cc(C(=O)N[C@@H](C)Cn2ccc(-c3ccc(C#N)c(Cl)c3)n2)n[nH]1]

Darolutamide is an oral non-steroidal androgen receptor (AR) inhibitor, which is indicated for treating patients with non-metastatic castration-resistant prostate cancer (nmCRPC). In 2019, Darolutamide developed by Bayer, is approved for market by the US Food and Drug Administration (FDA). In addition, a Phase III clinical trial of Darolutamide shows that compared to placebo combined with androgen deprivation therapy (ADT), Darolutamide combined with ADT significantly prolonged median metastasis free survival (MFS) (18.4 months vs. 40.4 months) and reduced the risk of disease metastasis or death by 59%, while also demonstrating good safety.

Darolutamide is a drug with low solubility and high permeability (BCS Class II) which is essentially insoluble in aqueous media at pH 1-6.8 (14-23 μg/mL). Single dose pharmacokinetic study shows that the median Tₘₐₓ of Darolutamide is 3-6 hours, indicating slow absorption. The end half-life of Darolutamide is 10-15 hours. Under fasting and feeding conditions, Cₘₐₓ and AUC₍₀₋₄₎ of a 600 mg dosage are higher than 300 mg. For the coefficient of variation (CV %) of AUC₍₀₋₄₎, the administration dosage of 300 mg (69.6%) is higher than that of 600 mg (41.4%).

In addition, the bioavailability of administration of 300 mg Darolutamide in fasting state is about 30%. In feeding state, the bioavailability of Darolutamide is increased by 2.5-fold and 2.8-fold after single dose administration of 300 mg or 600 mg, respectively. Similarly, in feeding state, the AUC₍₀₋₄₎ of Darolutamide after administration of 300 mg or 600 mg is increased by 2.5-fold. It can be seen that Darolutamide has a significant food effect.

WO2019032840A1 discloses a pharmaceutical composition comprising Abiraterone acetate and Darolutamide, and further comprising polyvinyl pyrrolidone or vinyl pyrrolidone/vinyl acetate copolymer as first pharmaceutical excipient and sodium lauryl sulfate as second pharmaceutical excipient. The inventors assert that the pharmaceutical composition has increased in vitro permeability under fasting and feeding conditions, to reduce drug dosage and avoid the limitation of empty stomach application without any example for proving such an effect.

### SUMMARY

In an aspect, provided is a pharmaceutical composition, comprising Darolutamide or a pharmaceutically acceptable salt thereof as active ingredient, a carrier material, and a formulation modifying agent; wherein the formulation modifying agent is one or more selected from the group consisting of: sodium lauryl sulfate, vitamin E polyethylene glycol succinate, Poloxamer, polyoxyethylene hydrogenated castor oil, octadecanol, dibutyl sebacate, triethyl citrate, butyl citrate, glycerol, polyethylene glycol, lecithin, dioctyl sulfosuccinate sodium, sodium taurocholate, polysorbitol, polyoxyethylene alkyl ether, polyoxyethylene fatty acid ester, and polyoxyethylene castor oil; the carrier material is one or more selected from the group consisting of: hydroxypropylmethyl cellulose phthalate, hydroxypropyl methylcellulose, polyvinyl alcohol, polyvinyl pyrrolidone, hydroxypropyl methyl cellulose acetate succinate, polyvinyl caprolactam-polyvinyl acetate-polyethylene glycol graft copolymer, Eudragit, copovidone, methyl cellulose, ethyl cellulose, sodium carboxymethyl cellulose, hydroxypropyl cellulose, polyvinyl acetate, cyclodextrin, sodium carboxymethyl ethyl cellulose polyethylene oxide, cellulose acetate phthalate, and cellulose acetate trimellitate.

In an embodiment, the weight ratio of Darolutamide or a pharmaceutically acceptable salt as active ingredient to the carrier material is about 1:0.5-1:8.

In an embodiment, the weight ratio of Darolutamide or a pharmaceutically acceptable salt as active ingredient to the formulation modifying agent is about 1:0.05-1:0.8.

In another aspect, provided is a solid formulation, comprising the pharmaceutical composition according to the present disclosure.

In another aspect, provided is a process for preparing the pharmaceutical composition according to the present disclosure, comprising the steps of (1) weighing each of the ingredients, and (2) mixing the ingredients in step (1) and subjecting the mixture to hot-melt extrusion, to obtain the pharmaceutical composition.

In another aspect, provided is a process for preparing the solid formulation according to the present disclosure, comprising the steps of (1) preparing a pharmaceutical composition according to the above-mentioned process, (2) pulverizing the pharmaceutical composition of step (1) to obtain pharmaceutical composition granules; (3) mixing the pharmaceutical composition granules of step (2) with a lubricant and other formulation modifying agent to obtain total mixed granules, (4) pressing the total mixed granules of step (3) to give the solid formulation.

In yet another aspect, provided is use of the pharmaceutical composition according to the present disclosure or the solid formulation according to the present disclosure for the manufacture of a medicament for preventing or treating prostate cancer.

### DETAILED DESCRIPTION

The present disclosure will be described in detail below, and the description is provided for the purpose of illustration rather than limitation. Those skilled in the art can easily understand the other advantages and efficacies of present disclosure according to the contents disclosed in present description. The present disclosure can also be implemented or applied through other specific embodiments. Those skilled in the art can perform various modifications and changes without deviation from the spirit of present disclosure.

### General Definition and Terms

Unless otherwise stated, all publication, patent applications, patents and other reference referred to herein are incorporated by reference in entirety.

Unless otherwise stated, the technical and scientific terms used herein have the same meaning as commonly understood by a person skilled in the art. If there is a contradiction, the definition provided in this application shall prevail.

Unless specified otherwise, all the percentages, portions and ratios, etc. in the present disclosure are on weight basis.

When an amount, concentration or other value or parameter is given as a range, a preferable range or a preferable upper limit and lower limit, or a specific value, it should be understood that it corresponds to specifically disclosure of any range by combining any pair of upper limit of any range or preferable value with the lower limit of any range or preferable value, regardless of whether the range is specifically disclosed. Unless otherwise stated, when numerical ranges are recited herein, the numerical ranges listed herein are intended to include the endpoints of the range and all integers and fractions within the range. A range in the present disclosure is not limited to the particular values cited when defining the range. For example, “1-20” encompasses 1, 2, 3, 4, 5, 6, 7, 8, 9, 10, 11, 12, 13, 14, 15, 16, 17, 18, 19, 20 and any subranges constituted from any two of these values, such as 2-6, 3-5, 2-10, 3-15, 4-20, 5-19 or the like.

The terms “about” and “approximately”, when used along with a numerical variable, generally means the value of the variable and all the values of the variable within an experimental error (e.g. 95% confidence interval for the mean) or within a specified value±10% or within a broader range.

The term “stoichiometric” means that substances are used in a certain weight ratio. For example, in the present disclosure, the active ingredient, the filler, the binder and the lubricant are formulated in a specified weight ratio.

The term “selected from” as used herein refers to one or more elements of the group listed thereafter, selected independently, and may encompass the combination of two or more elements.

The term “at least one” or “one or more” as used herein means one, two, three, four, five, six, seven, eight, nine or more.

Unless otherwise stated, the terms “a combination thereof” and “a mixture thereof” refer to a multi-component mixture of the elements, such as two, three, four and up to the maximum possible multi-component mixture.

In addition, if a component in the present disclosure is not indicated with a number, it indicates that there is no limit to the number of the component (or presence). Therefore, it should be interpreted as including one or at least one, and the singular form of a component also includes the plural, unless the value clearly indicates the singular.

The term “optional” or “optionally” mean that the subsequently described event or circumstance may or may not occur, and that the description includes the occurrence and non-occurrence of said event or circumstance.

The expressions “comprise” or the equivalents “include”, “contain” and “have” and the like are open-ended and do not exclude additional unrecited elements, steps or ingredients. Those skilled in the art will understand that the above-mentioned term “comprise” encompasses the meaning of “consisting of”. The expression “consisting of” excludes any element, step or ingredient not indicated. The expression “essentially consisting of” means that the scope is limited to the indicated elements, steps or ingredients, plus optional elements, steps or ingredients that do not materially affect the basic and novel characteristics of the claimed subject matter. It should be understood that the expression “comprise” encompasses the expressions “essentially consisting of” and “consisting of”.

The term “pharmaceutically acceptable” substance means a substance which, according to a common medical judgment, is suitable to be in contact with a tissue of a patient without any inappropriate toxicity, irritation, allergic response, etc., has a reasonable balance between advantages and disadvantages, and can be applied to its target use effectively. The term “pharmaceutically acceptable excipient” refers to a carrier substance which has no significant irritant effect on an organism and does not impair the biological activity and property of an active compound. The “pharmaceutically acceptable excipient” comprises but not limited to a glidant, a sweetener, a diluent, a preservative, a dye/colorant, a corrigent, a surfactant, a humectant, a dispersant, a disintegrant, a stabilizer, a solvent or an emulsifier. Non-limiting examples of the carrier comprise calcium carbonate, calcium phosphate, various sugars and various starches, cellulose derivate, gelatin, vegetable oil, polyethylene glycol and the like. Information regarding the carrier may be referred to Remington: The Science and Practice of Pharmacy, 21st Ed., Lippincott, Williams & Wilkins (2005), of which the content is incorporated herein by reference.

The terms “active pharmaceutical ingredient”, “active ingredient”, “therapeutic agent”, “active substance” or “active agent” refer to a chemical entity which is effective in treating or preventing a target disease or condition.

For pharmaceutical, pharmaceutical unit or active ingredient, the term “effective amount”, “therapeutically effective” or “prophylactically effective amount” refers to an amount of a pharmaceutical or agent that is sufficient to achieve the desired therapeutic effect under the conditions of acceptable side effects. The determination of an effective amount is individualized, depending on the age and general condition of the subject, as well as the specific active substance. The appropriate effective amount in a specific case may be determined by those skilled in the art according to conventional means.

The term “tablet” refers to a solid pharmaceutical dosage form prepared by compressing or molding techniques which comprises an active ingredient and optionally comprises a suitable excipient (e.g., a diluent, a binder or the like). Examples of the tablet comprises a compressed tablet, a multi-granule tablet, a multi-compressed tablet, a coated tablet, a matrix tablet, an osmotic pump tablet, a caplet or the like.

The term “bulk density” refers to the mass of a certain amount of granular product divided by the total volume occupied by the amount.

The term “angle of repose” refers to the maximum angle formed between the free slope formed by powder accumulation and the horizontal plane. The smaller the angle of repose, the smaller the friction and the better the fluidity.

The term “dissolution” refers to the rate and degree at which a drug dissolves from a solid formulation in a specified solvent.

### Pharmaceutical Composition

In an aspect, provided is a pharmaceutical composition, comprising Darolutamide or a pharmaceutically acceptable salt thereof as active ingredient, a carrier material, and a formulation modifying agent.

### Active Ingredient

In an embodiment, the active ingredient is Darolutamide or a pharmaceutically acceptable salt thereof. In a preferable embodiment, the active ingredient is Darolutamide.

### Carrier Material

The carrier material refers to a material generally in the form of particles, which is used to carry the active ingredient, wherein the active ingredient is substantially distributed within the carrier material.

In an embodiment, the carrier material is one or more selected from the group consisting of: hydroxypropylmethyl cellulose phthalate (HPMCP), hydroxypropyl methylcellulose (HPMC), polyvinyl alcohol (PVA), polyvinyl pyrrolidone (PVP), hydroxypropyl methyl cellulose acetate succinate (HPMCAS), polyvinyl caprolactam-polyvinyl acetate-polyethylene glycol graft copolymer (e.g., Soluplus®), Eudragit (e.g., EUDRAGIT®), copovidone, methyl cellulose, ethyl cellulose, sodium carboxymethyl cellulose, hydroxypropyl cellulose, polyvinyl acetate, cyclodextrin, sodium carboxymethyl ethyl cellulose polyethylene oxide, cellulose acetate phthalate, and cellulose acetate trimellitate. In a preferable embodiment, the carrier material is one or more selected from the group consisting of: hydroxypropylmethyl cellulose phthalate, hydroxypropyl methylcellulose, polyvinyl pyrrolidone, hydroxypropyl methyl cellulose acetate succinate, polyvinyl caprolactam-polyvinyl acetate-polyethylene glycol graft copolymer, and Eudragit. A suitable carrier material is conducive for increasing solubility of the pharmaceutical composition.

As an example, the carrier material that can be used comprises but not limited to AQOAT® (HPMCAS) commercially available from Shin-Etsu, Kollidon®RVA64 and SoluplusR commercially available from BASF, PVP K30 commercially available from Ashland.

### Formulation Modifying Agent

In an embodiment, the formulation modifying agent is one or more selected from the group consisting of: sodium lauryl sulfate, vitamin E polyethylene glycol succinate, Poloxamer, polyoxyethylene hydrogenated castor oil, octadecanol, dibutyl sebacate, triethyl citrate, butyl citrate, glycerol, polyethylene glycol, lecithin, dioctyl sulfosuccinate sodium, sodium taurocholate, polysorbitol, polyoxyethylene alkyl ether, polyoxyethylene fatty acid ester, and polyoxyethylene castor oil. In a preferable embodiment, the formulation modifying agent is one or more selected from the group consisting of: sodium lauryl sulfate, and vitamin E polyethylene glycol succinate, triethyl citrate.

As an example, the useful formulation modifying agent comprises but not limited to VE TPGS1000 commercially available from PMC Isochem, KolliphorRSLS commercially available from BASF, TEC commercially available from Merck KGaA.

In the field, sodium lauryl sulfate, vitamin E polyethylene glycol succinate, Poloxamer, polyoxyethylene hydrogenated castor oil, octadecanol, dibutyl sebacate, triethyl citrate, butyl citrate, glycerol, polyethylene glycol, lecithin, dioctyl sulfosuccinate sodium, sodium taurocholate, polysorbitol, polyoxyethylene alkyl ether, polyoxyethylene fatty acid ester, polyoxyethylene castor oil or the like is generally used as surfactant or plasticizer in a solid formulation, for example, to make the coating film of a coated tablet more flexible, so that the coated tablet is less prone to cracking, and easy to store and transport.

The present inventors surprisingly find that the formulation modifying agent used in the present disclosure can achieve unexpected technical effects, such as further improving the solubility of the pharmaceutical composition, thereby enhancing the bioavailability and stability of the drug, rendering pharmaceutical composition easy for preparation, thereby lowering energy consumption in production.

In an embodiment, in the pharmaceutical composition according to the present disclosure, when the formulation modifying agent is polyethylene glycol, the carrier material is not polyvinyl pyrrolidone and/or polyvinyl caprolactam-polyvinyl acetate-polyethylene glycol graft copolymer and/or hydroxypropyl cellulose.

In an embodiment, in the pharmaceutical composition according to the present disclosure, when the formulation modifying agent is sodium lauryl sulfate, the carrier material is not polyvinyl pyrrolidone and/or polyvinyl caprolactam-polyvinyl acetate-polyethylene glycol graft copolymer and/or hydroxypropyl cellulose.

In an embodiment, in the pharmaceutical composition according to the present disclosure, when the formulation modifying agent is Poloxamer, the carrier material is not polyvinyl pyrrolidone and/or polyvinyl caprolactam-polyvinyl acetate-polyethylene glycol graft copolymer and/or hydroxypropyl cellulose.

In an embodiment, in the pharmaceutical composition according to the present disclosure, when the formulation modifying agent is two or three of polyethylene glycol, sodium lauryl sulfate and Poloxamer, the carrier material is not polyvinyl pyrrolidone and/or polyvinyl caprolactam-polyvinyl acetate-polyethylene glycol graft copolymer and/or hydroxypropyl cellulose.

In an embodiment, in the pharmaceutical composition according to the present disclosure, when the formulation modifying agent is polyethylene glycol, sodium lauryl sulfate or Poloxamer, the carrier material is not polyvinyl pyrrolidone and/or polyvinyl caprolactam-polyvinyl acetate-polyethylene glycol graft copolymer and/or hydroxypropyl cellulose.

In other words, when the formulation modifying agent as used is polyethylene glycol, sodium lauryl sulfate, Poloxamer or a combination thereof, the carrier material is not polyvinyl pyrrolidone, nor polyvinyl caprolactam-polyvinyl acetate-polyethylene glycol graft copolymer, nor hydroxypropyl cellulose, nor a combination of two or three of them.

In a preferable embodiment, in the pharmaceutical composition according to the present disclosure, the formulation modifying agent is one or more selected from the group consisting of: sodium lauryl sulfate, vitamin E polyethylene glycol succinate, dibutyl sebacate, triethyl citrate, butyl citrate, glycerol, polyethylene glycol; the carrier material is one or more selected from the group consisting of: hydroxypropylmethyl cellulose phthalate, hydroxypropyl methylcellulose, polyvinyl alcohol, polyvinyl pyrrolidone, hydroxypropyl methyl cellulose acetate succinate, polyvinyl caprolactam-polyvinyl acetate-polyethylene glycol graft copolymer, Eudragit, copovidone, methyl cellulose, ethyl cellulose, sodium carboxymethyl cellulose; when the formulation modifying agent is sodium lauryl sulfate or polyethylene glycol, the carrier material is not polyvinyl pyrrolidone and/or polyvinyl caprolactam-polyvinyl acetate-polyethylene glycol graft copolymer.

In a more preferable embodiment, in the pharmaceutical composition according to the present disclosure, the formulation modifying agent is one or more selected from the group consisting of: sodium lauryl sulfate, vitamin E polyethylene glycol succinate, and triethyl citrate; the carrier material is one or more selected from the group consisting of: hydroxypropylmethyl cellulose phthalate, hydroxypropyl methylcellulose, polyvinyl pyrrolidone, hydroxypropyl methyl cellulose acetate succinate, polyvinyl caprolactam-polyvinyl acetate-polyethylene glycol graft copolymer, Eudragit; when the formulation modifying agent is sodium lauryl sulfate, the carrier material is not polyvinyl pyrrolidone and/or polyvinyl caprolactam-polyvinyl acetate-polyethylene glycol graft copolymer.

In a specific embodiment, in the pharmaceutical composition according to the present disclosure, the formulation modifying agent is vitamin E polyethylene glycol succinate, the carrier material is hydroxypropyl methyl cellulose acetate succinate. In a specific embodiment, in the pharmaceutical composition according to the present disclosure, the formulation modifying agent is polyvinyl pyrrolidone, the carrier material is hydroxypropyl methyl cellulose acetate succinate. In a specific embodiment, in the pharmaceutical composition according to the present disclosure, the formulation modifying agent is vitamin E polyethylene glycol succinate, the carrier material is hydroxypropyl methyl cellulose acetate succinate and polyvinyl pyrrolidone. In a specific embodiment, in the pharmaceutical composition according to the present disclosure, the formulation modifying agent is triethyl citrate, the carrier material is Eudragit.

### Other Ingredients

In an embodiment, the pharmaceutical composition according to the present disclosure may further comprises a pharmaceutically acceptable excipient.

In an embodiment, the pharmaceutical composition according to the present disclosure may further comprise one or more selected from the group consisting of: a buffer, an acidifier, a stabilizer, a preservative.

### Buffer

The buffer refers to a pharmaceutical excipient which stabilizes the pH of a pharmaceutical formulation.

In an embodiment, the buffer is one or more selected from the group consisting of: citric acid buffer, malate buffer, maleate buffer, and tartrate buffer.

### Acidifier

The acidifier refers to an acid which is mainly used as excipient in pharmaceutical preparation. It is used to regulate the pH of the drug and only provides acidity upon use, generally without introduction of any particular biological activity.

In an embodiment, the acidifier is one or more selected from the group consisting of: tartaric acid, carbonic acid, acetic acid, oxalic acid, and nitrous acid.

### Stabilizer

The stabilizer refers to a particular chemical substance which interacts with the active ingredient and/or common pharmaceutical excipient in the pharmaceutical composition to enhance the stability.

In an embodiment, the stabilizer is one or more selected from the group consisting of: methionine, lysine, and histidine.

### Preservative

The preservative refers to a compound which is added to the pharmaceutical composition for preventing or delaying microbial activity (growth and metabolism).

In an embodiment, the preservative is one or more selected from the group consisting of: benzyl alcohol, benzyl benzoate, methyl 4-hydroxybenzoate, propyl 4-hydroxybenzoate, vitamin E, and vitamin A palmitate.

In an embodiment, the weight ratio of Darolutamide or a pharmaceutically acceptable salt thereof as active ingredient to the carrier material is about 1:0.5-1:8. In a preferable embodiment, the weight ratio of Darolutamide or a pharmaceutically acceptable salt thereof as active ingredient to the carrier material is about 1:1-1:4. In a more preferable embodiment, the weight ratio of Darolutamide or a pharmaceutically acceptable salt thereof as active ingredient to the carrier material is about 1:2-1:4, for example, about 1:0.5, about 1:0.8, about 1:1, about 1:2, about 1:3, about 1:4, about 1:5, about 1:6, about 1:7, about 1:8, and a range composed of any two of these ratios (values). An appropriate weight ratio of the active ingredient to the carrier material is beneficial for obtaining suitable solubility and dissolution. An overly high weight ratio of the active ingredient to the carrier material cannot increase solubility of the active ingredient effectively; while an overly low weight ratio of the active ingredient to the carrier material cannot increase solubility of the active ingredient effectively either, which may lead to overly high viscosity and difficulty in preparation.

In an embodiment, the weight ratio of Darolutamide or a pharmaceutically acceptable salt thereof as active ingredient to the formulation modifying agent is about 1:0.05-1:0.8. In a preferable embodiment, the weight ratio of Darolutamide or a pharmaceutically acceptable salt thereof as active ingredient to the formulation modifying agent is about 1:0.1-1:0.5, for example, about 1:0.05, about 1:0.1, about 1:0.15, about 1:0.2, about 1:0.25, about 1:0.3, about 1:0.35, about 1:0.4, about 1:0.45, about 1:0.5, and a range composed of any two of these ratios (values). An appropriate weight ratio of the active ingredient to the formulation modifying agent is beneficial for obtaining suitable solubility, dissolution and stability, and the obtained drug is easy for storage and absorption by human. An overly high weight ratio of the active ingredient to the formulation modifying agent cannot increase solubility of the active ingredient effectively; while an overly low weight ratio of the active ingredient to the formulation modifying agent cannot increase solubility of the active ingredient effectively either and is incapable of preparation.

### Bulk Density

The bulk density refers to the mass of the pharmaceutical composition granules according to the present disclosure divided by the volume the granules occupy. An appropriate bulk density is beneficial for the preparation of the pharmaceutical composition and the formulation product, thereby facilitating the acquisition of the product with suitable solubility and dissolution.

In an embodiment, the pharmaceutical composition has a bulk density of about 0.40-0.50 g/mL. In a preferable embodiment, the pharmaceutical composition has a bulk density of about 0.42-0.49 g/mL. In a more preferable embodiment, the pharmaceutical composition has a bulk density of about 0.436-0.473 g/mL, for example about 0.40 g/mL, 0.41 g/mL, 0.42 g/mL, 0.423 g/mL, 0.425 g/mL, 0.427 g/mL, 0.429 g/mL, 0.43 g/mL, 0.433 g/mL, 0.436 g/mL, 0.439 g/mL, 0.44 g/mL, 0.442 g/mL, 0.445 g/mL, 0.448 g/mL, 0.45 g/mL, 0.451 g/mL, 0.46 g/mL, 0.466 g/mL, 0.469 g/mL, 0.47 g/mL, 0.473 g/mL, 0.48 g/mL, 0.49 g/mL.

### Angle of Repose

The angle of repose refers to the maximum angle formed between the free slope formed by the pharmaceutical composition granule according to the present disclosure and the horizontal plane. Wherein, the smaller the angle of repose, the smaller friction among the pharmaceutical composition granules according to the present disclosure, that is, the better fluidity of the pharmaceutical composition granules. An appropriate angle of repose of the pharmaceutical composition granules is beneficial for preparation of the formulation product, thereby facilitating the acquisition of the formulation product with suitable solubility and dissolution.

In an embodiment, the pharmaceutical composition has an angle of repose of about 20-32°. In a preferable embodiment, the pharmaceutical composition has an angle of repose of about 21-30°. In a more preferable embodiment, the pharmaceutical composition has an angle of repose of about 22-28°, for example about 20°, about 21°, about 22°, about 23°, about 24°, about 25°, about 26°, about 27°, about 28°, about 29°, about 30°, about 31°, about 32°.

### Solid Formulation

In another aspect, provided is a solid formulation, comprising the pharmaceutical composition according to the present disclosure.

### Preparation Process According to the Present Disclosure

### Preparation of the Pharmaceutical Composition

In another aspect, provided is a process for preparing the pharmaceutical composition according to the present disclosure, comprising the steps of:
(1) weighing each of the ingredients, and
(2) mixing the ingredients in step (1) and subjecting the mixture to hot-melt extrusion, to obtain the pharmaceutical composition.

The active ingredient, carrier material and formulation modifying agent are as define above.

### Step (1)

Each of the ingredients of the pharmaceutical composition are weighed according to a particular stoichiometric ratio (e.g., weight ratio). For example, each of the ingredients of the pharmaceutical composition may be weighed according to the weight ratios in the following examples.

### Step (2)

The hot-melt extrusion in step (2) may be conducted with conventional methods and equipments in the field. For example, the hot-melt extrusion may be conducted on the ingredients in step (1) with Leistritz ZSE 12HP-PH-40D.

In an embodiment, the hot-melt extrusion in step (2) may be conducted at a temperature of about 160+20° C.

### Preparation of the Solid Formulation

In another aspect, provided is also a process for preparing the solid formulation, comprising the steps of
(1) preparing a pharmaceutical composition according to the above process,
(2) pulverizing the pharmaceutical composition of step (1) to obtain pharmaceutical composition granules,
(3) mixing the pharmaceutical composition granules of step (2) with a lubricant and other formulation modifying agent to obtain total mixed granules,
(4) pressing the total mixed granules to obtain the solid formulation.

### Step (2)

The pulverization in step (2) may be conducted with conventional methods in the field. For example, blade pulverization and roller pulverization may be used.

### Step (3)

The mixing in step (3) may be conducted with conventional methods in the field. For example, V-shaped mixing and hopper mixing may be used.

### Step (4)

The pressing in step (4) may be conducted with conventional methods in the field. For example, hydraulic pressing, mechanical pressing, atmospheric pressure or the like may be used. The useful equipment may be ZP14 press from Gylongli Sci.&Tech.Co., Ltd. or the like. In an embodiment, preparation of the solid formulation according to the present disclosure further comprises a step of:

Step (5) coating the solid formulation of step (4) to obtain a tablet.

The coating in step (5) may be conducted with conventional methods in the field. For example, Ohara coating machine or the like may be used.

It should be noted that the listed preparation procedures for the composition are illustrative and exemplary only, rather than exhaustive. Accordingly, the preparation procedures of the composition are note limited to those as listed above. Those skilled can make various changes, modifications or equivalents to the preparation procedures with conventional technology, which all fall within the scope of the present disclosure.

### Pharmaceutical Use

Provided is also a method for preventing and treating prostate cancer, comprising administering a subject in need thereof a therapeutically effective amount of the pharmaceutical composition or solid formulation according to the present disclosure.

Provided is also the pharmaceutical composition or solid formulation according to the present disclosure for use in preventing and treating prostate cancer.

Provided is also use of the pharmaceutical composition or the solid formulation according to the present disclosure for the manufacture of a medicament for preventing and treating prostate cancer.

### Beneficial Effects

Compared to the Darolutamide pharmaceutical composition in the prior art, the Darolutamide pharmaceutical composition according to the present disclosure can further increase the solubility of the active ingredient, thereby obtaining the pharmaceutical composition with excellent solubility, and increase the bioavailability of the composition. Accordingly, the Darolutamide pharmaceutical composition according to the present disclosure can be administered at low dosage and reduce the food effect.

In addition, the Darolutamide pharmaceutical composition according to the present disclosure has excellent stability and is suitable for storage at ambient temperature, lowering the requirements for storage and transport.

Further, the Darolutamide pharmaceutical composition according to the present disclosure has a suitable bulk density and a suitable angle of repose, showing good fluidity and thus is conducive for preparation into the forms like tablet and suitable for large scale and industrial productions.

Moreover, by using a suitable carrier material and a suitable formulation modifying agent, compared to the prior art, the Darolutamide pharmaceutical composition according to the present disclosure shows significantly increased solubility, dissolution and stability. The obtained pharmaceutical composition granules have improved bulk density and angle of repose and are more conducive for operations like pressing, to prepare the solid formulation, whereby the production energy consumption is reduced effectively.

### EXAMPLES

The technical solutions of the present disclosure will be further described below by reference to specific examples.

It should be noted that the following examples are only examples for clearly explaining the technical solutions of the present disclosure rather than limitations thereto. For those skilled in the art, other changes or modifications in different forms can be made on the basis of the above description, and it is unnecessary and impossible to exhaust all the embodiments herein and the obvious changes or modifications derived therefrom are still within the protection scope of the present disclosure. Unless otherwise stated, the apparatus, reagents, raw materials or the like herein are commercially available.

### Materials

Darolutamide: Purchased from Shanghai Pengpai Biotechnology Co., Ltd;
PVP: Purchased from BASF, Kollidon VA64;
   Purchased from Ashland, PVP K30;
HPMCAS: Purchased from Shin-Etsu, AQOATR;
HPMCP: Purchased from Shin-Etsu, hydroxypropylmethyl cellulose phthalate;
Polyvinyl caprolactam-polyvinyl acetate-polyethylene glycol graft copolymer: Purchased from BASF, SoluplusR
Eudragit: Purchased from Evonik Degussa, EUDRAGIT®;
VE TPGS: Purchased from PMC Isochem, VE TPGS1000;
SLS: Purchased from BASF, Kolliphor®;
TEC: Purchased from Merck KGAA, TEC.

### Determination of Solubility

About 200 mg of sample is weighted into a 50 ml shaking flask and then 50 ml of medium with pH of 6.8 is added. The shaking flask is mixed well and placed in a shaker. The shaker temperature is set as 37.2° C. and the rotation speed is set as 50 rpm. The sample is taken at 4 hours and 0.45 μM filter membrane is used for filtration to collect filtrate. High-performance liquid chromatography is used for determination with wavelength at 286 nm.

### Determination of Dissolution

In the following examples, the dissolution of the formulation is determined using the USP II method (paddle method) in the US Pharmacopoeia, with specific parameters as follows.

| Dissolution method | USP II method (paddle method) | |
|---|---|---|
| Dissolution medium | phosphate buffer, pH 6.8 | |
| Medium volume | 1000 | ml |
| Rotation speed | 75 | rpm |

| | |
|---|---|
| Temperature | 37° C. ± 5° C. |

Dissolved sample analysis: The solution obtained in the dissolution test is filtered with 0.45 μm membrane to collect the filtrate and High-performance liquid chromatography is used for determination with wavelength at 260 nm.

Content test method: The test sample is dissolved in methanol to give the concentration of 100 μg/ml of Darolutamide, and High-performance liquid chromatography is used for determination with wavelength at 286 nm.

Related substances test method: The test sample is dissolved in methanol to give the concentration of 150 μg/ml of Darolutamide, and High-performance liquid chromatography is used for determination with wavelength at 286 nm.

### PREPARATION EXAMPLES

### Example 1. Preparation of Darolutamide Pharmaceutical Composition

Darolutamide pharmaceutical composition granules were prepared according to the ingredients and weight ratios in Table 1.

The Preparation Procedures were as Follows:

The active ingredient and carrier material were mixed according to the weight ratios in Table 1 to obtain the blend. The blend was added to Leistritz ZSE 12HP-PH-40D hot-melt extruder at a constant rate via a powder feeder and hot melt extrusion was conducted at a temperature of 160+20° C. The extrudate was collected and pulverized using FitzMill LIA pulverizer. The pulverize extrudate was sieved through 80 mesh screen.

According to the above procedures, Darolutamide Pharmaceutical composition 1-1 to 1-15 and Blend 1-1 to 1-15 of active ingredient and carrier material were obtained, respectively.

TABLE 1
Ingredients of Darolutamide Compositions

| | | | Active |
|---|---|---|---|
| | Active | Carrier | ingredient:Carrier |
| Composition | ingredient | material | material |
| Composition 1-1 | Darolutamide | VA64 | 1:2 |
| Composition 1-2 | Darolutamide | VA64 | 1:3 |
| Composition 1-3 | Darolutamide | VA64 | 1:4 |
| Composition 1-4 | Darolutamide | HPMCAS | 1:1 |
| Composition 1-5 | Darolutamide | HPMCAS | 1:2 |
| Composition 1-6 | Darolutamide | HPMCAS | 1:3 |
| Composition 1-7 | Darolutamide | HPMCAS | 1:4 |
| Composition 1-8 | Darolutamide | HPMCP-HP55 | 1:2 |
| Composition 1-9 | Darolutamide | HPMCP-HP55 | 1:3 |
| Composition 1-10 | Darolutamide | Soluplus | 1:2 |
| Composition 1-11 | Darolutamide | Soluplus | 1:3 |
| Composition 1-12 | Darolutamide | Eudragit-L100 | 1:2 |
| Composition 1-13 | Darolutamide | Eudragit-L100 | 1:3 |
| Composition 1-14 | Darolutamide | PVP K30 | 1:3 |
| Composition 1-15 | Darolutamide | PVP K30 | 1:4 |

### Example 2. Preparation of Darolutamide Pharmaceutical Composition According to the Present Disclosure

Darolutamide pharmaceutical composition granules were prepared according to the ingredients and weight ratios in Table 2.

The Preparation Procedures were as Follows:

The active ingredient, carrier material and formulation modifying agent were mixed according to the weight ratios in Table 2 to obtain the blend. The blend was added to Leistritz ZSE 12HP-PH-40D hot-melt extruder at a constant rate via a powder feeder and hot melt extrusion was conducted at a temperature of 160+20° C. The extrudate was collected and pulverized using FitzMill LIA pulverizer. The pulverize extrudate was sieved through 80 mesh screen.

According to the above procedures, Darolutamide Pharmaceutical composition 2-1 to 2-9 and Blends 2-1 to 2-9 of active ingredient, carrier material and formulation modifying agent were obtained, respectively.

TABLE 2
Ingredients of Darolutamide Compositions

| | | | | Mass ratio (Active |
|---|---|---|---|---|
| | | | Formulation | ingredient:Carrier |
| | Active | | modifying | material:Formulation |
| Composition | ingredient | Carrier material | agent | modifying agent) |
| Composition 2-1 | Darolutamide | HPMCAS | TPGS | 1:2:0.2 |
| Composition 2-2 | Darolutamide | HPMCAS | TPGS | 1:4:0.5 |
| Composition 2-3 | Darolutamide | VA64 + HPMCAS | / | 1:0.5:1.5 |
| Composition 2-4 | Darolutamide | VA64 + HPMCAS | TPGS | 1:0.5:1.5:0.2 |
| Composition 2-5 | Darolutamide | HPMCP-HP55 | TPGS | 1:3:0.1 |
| Composition 2-6 | Darolutamide | Eudragit L100 | TEC | 1:2:0.15 |
| Composition 2-7 | Darolutamide | Soluplus | SLS | 1:3:0.1 |
| Composition 2-8 | Darolutamide | PVP K30 | SLS | 1:4:0.5 |
| Composition 2-9 | Darolutamide | VA64 | SLS | 1:4:0.5 |

### Example 3 Preparation of Darolutamide Tablet

Darolutamide tablets were prepared according to the ingredients and weight ratios in Table 3.

The Preparation Procedures of Darolutamide Tablet were as Follows:

(1) Darolutamide pharmaceutical compositions were prepared according to the procedures in Example 1 and Example 2, respectively. (2) The pharmaceutical compositions in step (1) were mixed with filler, disintegrant and lubricant to obtain the total mixed granules. (3) The total mixed granules in step (2) were pressed with ZP14 tablet pressing machine to obtain Darolutamide tablets.

TABLE 3
Ingredients of Darolutamide tablets

| | Function | Tablet 2-1 | Tablet 2-2 | Tablet 2-3 | Tablet 2-4 | Tablet 2-5 | Tablet 2-6 |
|---|---|---|---|---|---|---|---|
| Composition 1-5 | — | 900 | — | — | — | — | — |
| Composition 2-1 | — | — | 960 | — | — | — | — |
| Composition 2-4 | — | — | — | 960 | — | — | — |
| Composition 2-6 | — | — | — | — | 945 | — | — |
| Composition 2-8 | — | — | — | — | — | 1650 | — |
| Composition 2-9 | — | — | — | — | — | — | 1650 |
| Microcrystalline cellulose | filler | 300 | 240 | 240 | 255 | 230 | 230 |
| Croscarmellose sodium | disintegrant | 74 | 74 | 74 | 74 | 90 | 90 |
| Magnesium stearate | lubricant | 26 | 26 | 26 | 26 | 30 | 30 |

### TESTING EXAMPLES

### Example 4 Crystal Form Test of Pharmaceutical Composition

Crystal form determination of the pharmaceutical compositions comprised polycrystalline Powder X-ray Diffraction (PXRD) and Differential Scanning calorimetry (DSC), which were determined using Bruker D8 advance (scan range:) 3°−40° and TA Instruments DSC2000 (scan range: 330° C.-250° C.), respectively.

TABLE 4
Darolutamide Composition Crystal Form

| Composition | Crystal form |
|---|---|
| Composition 1-1 | partially amorphous form |
| Composition 1-2 | amorphous form |
| Composition 1-3 | amorphous form |
| Composition 1-4 | partially amorphous form |
| Composition 1-5 | amorphous form |
| Composition 1-6 | amorphous form |
| Composition 1-7 | amorphous form |
| Composition 1-8 | amorphous form |
| Composition 1-9 | amorphous form |
| Composition 1-10 | partially amorphous form |
| Composition 1-11 | amorphous form |
| Composition 1-12 | amorphous form |
| Composition 1-13 | amorphous form |
| Composition 1-14 | amorphous form |
| Composition 1-15 | amorphous form |
| Composition 2-1 | amorphous form |
| Composition 2-2 | amorphous form |
| Composition 2-3 | amorphous form |
| Composition 2-4 | amorphous form |
| Composition 2-5 | amorphous form |
| Composition 2-6 | amorphous form |
| Composition 2-7 | amorphous form |
| Composition 2-8 | amorphous form |
| Composition 2-9 | amorphous form |

According to the crystal form testing results in Table 4, the pharmaceutical compositions prepared from the active ingredient Darolutamide and carrier material could form particles in amorphous form, increasing the solubility of Darolutamide. Meanwhile, the pharmaceutical composition granules are easy for pharmaceutical preparation, which was conducive for further preparation into solid formulations and for large-scale and industrial production.

### Example 5 Solubility Test of the Pharmaceutical Composition and the Blend

The solubilities of pharmaceutical composition 1-1 to 1-15, 2-1 to 2-9 and corresponding blends were tested according to the above-mentioned procedures, and the solubilities were as shown in Table 4. In addition, under the above experimental conditions, the solubility of Darolutamide compound was tested as 24.9 μg/ml.

TABLE 5
Solubilities of pharmaceutical compositions and blends

| | Pharmaceutical | Solubility of |
|---|---|---|
| Pharmaceutical | composition | corresponding |
| composition/blend | solubility (μg/ml) | blend (μg/ml) |
| Composition 1-1 | 107.2 | 23.9 |
| Composition 1-2 | 260.9 | 24.6 |
| Composition 1-3 | 328.5 | 26.3 |
| Composition 1-4 | 142.7 | 23.4 |
| Composition 1-5 | 488.9 | 24.1 |
| Composition 1-6 | 405.6 | 25.3 |
| Composition 1-7 | 421.6 | 24.9 |
| Composition 1-8 | 438.1 | 23.5 |
| Composition 1-9 | 406.2 | 23.1 |
| Composition 1-10 | 369.2 | 24.6 |
| Composition 1-11 | 307.5 | 24.7 |
| Composition 1-12 | 470.5 | 23.2 |
| Composition 1-13 | 490.8 | 25.9 |
| Composition 1-14 | 321.6 | 24.7 |
| Composition 1-15 | 394.2 | 26.8 |
| Composition 2-1 | 499.6 | 26.2 |
| Composition 2-2 | 462.3 | 28.1 |
| Composition 2-3 | 431.2 | 24.3 |
| Composition 2-4 | 479.1 | 25.3 |
| Composition 2-5 | 427.2 | 24.1 |
| Composition 2-6 | 538.2 | 24.8 |
| Composition 2-7 | 395.3 | 28.7 |
| Composition 2-8 | 416.5 | 33.5 |
| Composition 2-9 | 389.6 | 29.6 |

According to the solubility testing results in Table 5, with combination of active ingredient Darolutamide and carrier material, the obtained Darolutamide pharmaceutical composition showed significantly increased solubility. Compared to Darolutamide compound (solubility of 24.9 μg/ml) and its blend with carrier material and/or formulation modifying agent, the Darolutamide Composition showed significantly increased solubility of 107.2-490.8 μg/ml.

Based on Composition 1-1 to 1-15, the formulation modifying agent like TPGS, SLS or TEC was further added into the Darolutamide pharmaceutical composition and the Darolutamide pharmaceutical composition according to the present disclosure could show further increased solubility of 389.6-538.2 μg/ml. It can be seen that, addition of formulation modifying agent according to the present disclosure in the pharmaceutical composition could further increase solubility of the Darolutamide pharmaceutical composition.

In addition, in Composition 2-8 and Composition 2-9, polyvinyl pyrrolidone was used as carrier material, and sodium lauryl sulfate was used as formulation modifying agent. Compared to Composition 2-7 to Composition 2-9, Composition 2-1 to 2-2 and Composition 2-4 to Composition 2-6 showed significantly increased solubility. Accordingly, the selection of carrier material and formulation modifying agent could affect solubility of the pharmaceutical composition and a suitable selection was conducive for further increase of solubility of the pharmaceutical composition.

### Example 6 Bulk Density Test of the Pharmaceutical Composition

The bulk densities of the pharmaceutical compositions were determined with SOTAX TD2 bulk density device. The testing results were shown in Table 6.

TABLE 6
Bulk densities of pharmaceutical compositions

| Composition | Bulk density (g/mL) |
|---|---|
| Composition 1-1 | 0.384 |
| Composition 1-2 | 0.426 |
| Composition 1-3 | 0.413 |
| Composition 1-4 | 0.411 |
| Composition 1-5 | 0.433 |
| Composition 1-6 | 0.447 |
| Composition 1-7 | 0.461 |
| Composition 1-8 | 0.441 |
| Composition 1-9 | 0.452 |
| Composition 1-10 | 0.428 |
| Composition 1-11 | 0.446 |
| Composition 1-12 | 0.408 |
| Composition 1-13 | 0.415 |
| Composition 1-14 | 0.431 |
| Composition 1-15 | 0.419 |
| Composition 2-1 | 0.451 |
| Composition 2-2 | 0.466 |
| Composition 2-3 | 0.429 |
| Composition 2-4 | 0.473 |
| Composition 2-5 | 0.469 |
| Composition 2-6 | 0.436 |
| Composition 2-7 | 0.431 |
| Composition 2-8 | 0.429 |
| Composition 2-9 | 0.432 |

According to the bulk density testing results in Table 6, compared to Composition 1-1 to 1-15 (bulk density of 0.384-0.461 g/mL), Composition 2-1 to 2-9 showed relatively higher bulk densities of 0.429-0.473 g/mL. Accordingly, addition of formulation modifying agent in the pharmaceutical composition could further increase bulk density of the pharmaceutical composition, and the obtained pharmaceutical composition granules were suitable for pressing of tablet products, which could lower energy consumptions in production.

### Example 7 Angle of Repose Determination of the Pharmaceutical Compositions

The angles of repose of the pharmaceutical compositions were determined with Granutools Granuheap angle of repose analyzer. The results were shown in Table 7.

TABLE 7
Angles of repose of the pharmaceutical compositions

| Composition | Angle of repose (°) |
|---|---|
| Composition 1-1 | 45 |
| Composition 1-2 | 35 |
| Composition 1-3 | 34 |
| Composition 1-4 | 37 |
| Composition 1-5 | 32 |
| Composition 1-6 | 31 |
| Composition 1-7 | 29 |
| Composition 1-8 | 29 |
| Composition 1-9 | 31 |
| Composition 1-10 | 33 |
| Composition 1-11 | 32 |
| Composition 1-12 | 38 |
| Composition 1-13 | 34 |
| Composition 1-14 | 34 |
| Composition 1-15 | 33 |
| Composition 2-1 | 27 |
| Composition 2-2 | 27 |
| Composition 2-3 | 37 |
| Composition 2-4 | 22 |
| Composition 2-5 | 28 |
| Composition 2-6 | 28 |
| Composition 2-7 | 31 |
| Composition 2-8 | 32 |
| Composition 2-9 | 31 |

According to the testing results in Table 7, compared to Composition 1-1 to 1-15 (angle of repose of) 29-45°, Composition 2-1 to 2-9 showed relatively lower angles of repose of 22-37°. Accordingly, addition of formulation modifying agent in the pharmaceutical composition could further lower angle of repose of the composition, improving fluidity of the composition, and the obtained pharmaceutical composition granules were suitable for pressing of tablet products, which could lower energy consumptions in production.

In addition, Compared to Composition 2-8 and Composition 2-9, Composition 2-1 to 2-2 and Composition 2-4 to Composition 2-6 showed relatively lower angles of repose. Accordingly, the selection of carrier material and formulation modifying agent could affect angle of repose of the pharmaceutical composition and suitable carrier material and formulation modifying agent were conducive for relatively lower angle of repose of the composition, thereby further improving fluidity of the composition, and the obtained pharmaceutical composition granules were suitable for preparation of formulation products, which could lower energy consumptions in production.

### Example 8 Stability Test of the Pharmaceutical Composition

During storage, the pharmaceutical composition was prone to crystallization and an increase of related substances, resulting in unqualified product due to influence of environment (such as temperature, humidity or the like) on the formulation. To evaluate the stability of Darolutamide pharmaceutical composition, the stability was tested under 40° C./75% RH accelerated condition. The testing procedures were as follows:

Darolutamide pharmaceutical composition was fed into a HDPE bottle, which was placed in a stability box. Stability drug was taken at given time points for testing to give crystal form state of the composition, composition contents and related substances contents. The data was shown in Table 8.

TABLE 8
Darolutamide Composition Stability

| | Composition crystal form state/Composition content/Related substances content | | | |
|---|---|---|---|---|
| | | 40° C./75% RH- | 40° C./75% RH- | 40° C./75% RH- |
| Sample | Initial -T0 | 1 month | 3 months | 6 months |
| Composition 1-5 | amorphous form/ | amorphous form/ | amorphous form/ | amorphous form/ |
| | 97.4%/1.40% | 96.7%/1.52% | 95.1%/1.97% | 90.2%/2.68% |
| Composition 2-1 | amorphous form/ | amorphous form/ | amorphous form/ | amorphous form/ |
| | 97.6%/0.68% | 98.3%/0.72% | 97.3%/1.26% | 97.1%/1.74% |
| Composition 2-3 | amorphous form/ | — | — | — |
| | 90.1%/4.59% | | | |
| Composition 2-4 | amorphous form/ | amorphous form/ | amorphous form/ | amorphous form/ |
| | 96.1%/1.19% | 96.8%/1.37% | 95.6%/1.81% | 96.3%/1.94% |
| Composition 2-5 | amorphous form/ | amorphous form/ | amorphous form/ | amorphous form/ |
| | 97.3%/1.02% | 97.2%/1.69% | 96.6%/2.21% | 96.9%/4.14% |
| Composition 2-6 | amorphous form/ | amorphous form/ | amorphous form/ | amorphous form/ |
| | 96.1%/0.85% | 97.5%/1.13% | 96.8%/1.16% | 95.3%/1.78% |
| Composition 2-7 | amorphous form/ | amorphous form/ | amorphous form/ | amorphous form/ |
| | 98.3%/1.55% | 97.6%/1.97% | 97.5%/2.46% | 96.9%/3.07% |
| Composition 2-8 | amorphous form/ | amorphous form/ | amorphous form/ | amorphous form/ |
| | 98.7%/0.93% | 99.1%/1.32% | 97.3%/1.85% | 96.1%/2.68% |
| Composition 2-9 | amorphous form/ | amorphous form/ | amorphous form/ | amorphous form/ |
| | 99.5%/1.25% | 96.3%/1.58% | 97.1%/1.96% | 94.2%/3.21% |

According to the stability testing results in Table 8, after storage for 6 months, Composition 2-1, Composition 2-4 to Composition 2-9 showed contents higher than 94.2%, which was significantly higher than that of Composition 1-5.

Accordingly, compared to Darolutamide Composition pharmaceutical composition 1-5, with addition of formulation modifying agent into the pharmaceutical composition, the obtained pharmaceutical composition 2-1, Composition 2-4 to 2-9 showed significantly improved stability, effectively reducing generation of related substances. It can be seen that, by addition of formulation modifying agent according to the present disclosure into Darolutamide pharmaceutical composition, the obtained pharmaceutical composition showed significantly improved stability.

In addition, compared to Composition 2-7 to Composition 2-9, after storage for 6 months, Composition 2-1 and Composition 2-4 to Composition 2-6 showed contents higher than 96.3%, while those of Composition 2-7 to Composition 2-9 were lower than 96.1%. Accordingly, the selection of carrier material and formulation modifying agent could also affect stability of the pharmaceutical composition and suitable carrier material and formulation modifying agent were conducive for obtaining compositions with higher stability.

Moreover, the present inventors surprisingly found that when HPMCAS was used as carrier in the Darolutamide Composition, and formulation modifying agent TPGS was added into the pharmaceutical composition, the pharmaceutical composition could show relatively higher stability. For example, under the condition of 40° C./75% RH, after 6 months, the pharmaceutical composition showed the content as high as 97.1%.

### Example 9 Dissolution Test of the Pharmaceutical Composition

According to the above procedures, the dissolutions of Darolutamide in phosphate buffer, pH 6.8 were determined and the results were shown in Table 9.

TABLE 9
Dissolutions of Darolutamide tablets
Dissolution (%)

| Time point (min) | 5 | 15 | 30 | 45 |
|---|---|---|---|---|
| Tablet 2-1 | 47 | 81 | 93 | 92 |
| Tablet 2-2 | 51 | 90 | 94 | 94 |
| Tablet 2-3 | 56 | 89 | 96 | 97 |
| Tablet 2-4 | 45 | 77 | 92 | 95 |
| Tablet 2-5 | 41 | 72 | 94 | 93 |
| Tablet 2-6 | 39 | 75 | 94 | 94 |
| Darolutamide API | 7 | 10 | 9 | 12 |
| Darolutamide tablet (NUBEQA) | 4 | 8 | 9 | 10 |

According to the dissolution testing results in Table 9, compared to Darolutamide API and commercially available Darolutamide tablet (NUBEQA), the Darolutamide pharmaceutical composition tablet according to the present disclosure showed significantly improved dissolution and the dissolution rate and amount of Darolutamide were clearly increased. For example, under the experimental conditions, in phosphate buffer, pH 6.8, the Darolutamide tablets according to the present disclosure could release 90% of Darolutamide after 30 minutes and up to 97% of Darolutamide after 45 minutes.

Accordingly, with further addition of formulation modifying agent into Darolutamide and the pharmaceutical composition thereof, the Darolutamide pharmaceutical composition according to the present disclosure could show significantly improved drug dissolution.

### Example 10 Pharmacokinetic Study of Darolutamide Tablet

### 1. Reagents

Test formulation T: Darolutamide tablet prepared according to formulation and procedure of tablet 2-3 in Example 3 with the specification of 300 mg/tablet.

Reference formulation R: Commercially available Darolutamide tablet (NUBEQA, with the specification of 300 mg/tablet)

### 2. EXPERIMENTAL PROCEDURES

### 2.1 Experiment 1

An open, randomized, double-period, double-crossover self-controlled trial design procedure (7-day washout period) was used, where 10 healthy male subjects were randomly divided into 2 groups, 5 in each group. The grouping design was shown in the table below:

TABLE 10
Grouping

| Period | Group 1 | Group 2 |
|---|---|---|
| Period I | T | R |
| Period II | R | T |

The subjects entered the Phase I clinical trial ward before the testing day, were given a uniform light diet in the evening, and then fasted overnight (at least 10 h, free for getting water). The subjects in the next morning around 8:00 am were each given high-fat meals then a tablet of Test formulation T or Reference formulation R, with 240 mL of water. No water was allowed for 2 h after administration with the upper body upright. The lunches were given after 4 h.

Blood sample collection procedure: 0 h before administration (within 1 h before administration) and 0.5, 1, 1.5, 2, 2.5, 3, 3.5, 4, 4.5, 5, 6, 7, 8, 12, 24, 36 h after administration, 4 mL of cubital venous blood was collected, and the blood samples were placed in blood collection tubes anticoagulated with heparin sodium. After collection, the samples were centrifuged at 3500 rpm for 5 min at 4° C. The plasma was divided into two parts, 1 mL of plasma was added to the formal tube and the remaining plasma was added to the backup tube. The samples were stored in a −20° C. refrigerator within 2 h after collection for subsequent use.

The LC-MS/MS method was used to determine the concentration of Darolutamide in each of the plasma samples.

### 2.2 Experiment 2

An open, randomized, double-period, double-crossover self-controlled trial design procedure (7-day washout period) was used, where 10 healthy male subjects were randomly divided into 2 groups, 5 in each group. The grouping design was shown in the table below:

TABLE 11
Grouping

| Period | Group 1 | Group 2 |
|---|---|---|
| Period I | Empty stomach | After meal |
| Period II | After meal | Empty stomach |

The subjects entered the Phase I clinical trial ward before the testing day, were given a uniform light diet in the evening, and then fasted overnight (at least 10 h, free for getting water). The subjects in the next morning around 8:00 am were each given high-fat meals then a tablet of Test formulation T, or each given a tablet of Test formulation T on an empty stomach, with 240 mL of water. No water was allowed for 2 h after administration with the upper body upright. The lunches were given after 4 h.

Blood sample collection procedure: 0 h before administration (within 1 h before administration) and 0.5, 1, 1.5, 2, 2.5, 3, 3.5, 4, 4.5, 5, 6, 7, 8, 12, 24, 36 h after administration, 4 mL of cubital venous blood was collected, and the blood samples were placed in blood collection tubes anticoagulated with heparin sodium. After collection, the samples were centrifuged at 3500 rpm for 5 min at 4° C. The plasma was divided into two parts, 1 mL of plasma was added to the formal tube and the remaining plasma was added to the backup tube. The samples were stored in a −20° C. refrigerator within 2 h after collection for subsequent use.

The LC-MS/MS method was used to determine the concentration of Darolutamide in each of the plasma samples.

### 3. Experimental Results

The results of Experiment 1 and 2 were summarized as follows.

TABLE 12
Pharmacokinetic data of Experiment 1

| | Test formulation | Reference formulation | |
|---|---|---|---|
| Pharmacokinetic | T, after meal | R, after meal | T/R |
| parameters (unit) | (mean ± SD) | (mean ± SD) | (%) |
| Tₘₐₓ (h) | 3.50 (2.00-5.00) | 3.00 (1.50-4.50) | / |
| Cₘₐₓ (ng/mL) | 1426.52 ± 451.23 | 984.28 ± 296.36 | 144.93 |
| AUC₀₋ₜ | 12122.75 ± 3175.12 | 10945.50 ± 2845.69 | 110.76 |
| (h*ng/mL) | | | |

TABLE 13
Pharmacokinetic data of Experiment 2

| | Test formulation T, | Test formulation T, | |
|---|---|---|---|
| Pharmacokinetic | empty stomach T1 | after meal T2 | T2/T1 |
| parameters (unit) | (mean ± SD) | (mean ± SD) | (%) |
| Tₘₐₓ (h) | 1.50 (1.00-4.00) | 3.00 (2.00-5.00) | / |
| Cₘₐₓ (ng/mL) | 1398.66 ± 423.54 | 1401.28 ± 472.63 | 100.19 |
| AUC₀₋ₜ (h*ng/mL) | 10917.25 ± 3436.58 | 11725.68 ± 3425.52 | 107.41 |

The data of Experiment 1 (Table 12) showed:
(1) The maximum plasma concentration Cₘₐₓ after administration of Test formulation T was increased significantly, which was 144.93% of that after administration of Reference formulation R.
(2) Compared to the area under the plasma concentration curve after administration of Reference formulation R, the area under the plasma concentration curve AUC₀₋₄ after administration of Test formulation T was increased by about 10%, which was 110.76% of that after administration of Reference formulation R.
(3) Compared to the plasma concentration time to peak after administration of Reference formulation R, the plasma concentration time to peak Tₘₐₓ after administration of Test formulation T did not show significant change.

The above results showed that, under the condition after meal, compared to Reference formulation R, Test formulation T showed improvement of absorption to some extent in the subjects.

### The Data of Experiment 2 (Table 13) Showed:

(1) Compared to the maximum plasma concentration Cₘₐₓ under the condition of empty stomach, the maximum plasma concentration Cₘₐₓ of Test formulation T after meal did not show significant change.
(2) Compared to the area under the plasma concentration curve under the condition of empty stomach, the area under the plasma concentration curve AUC₀₋₄ of Test formulation T after meal was increased by about 7%.
(3) Compared to the plasma concentration time to peak under the condition of empty stomach, the plasma concentration time to peak Tₘₐₓ of Test formulation T after meal was significantly postponed, which is mainly due to food effect.

Moreover, the instructions of Reference formulation R showed that its absorption under the condition of empty stomach was only 40%-50% of that after meal, and thus it should be administered after meal. However, the results of Experiment 2 showed that, the absorption of Test formulation T in body is substantially not affected by the food effect.

Accordingly, the Darolutamide tablet prepared according to the present disclosure improved the bioavailability of the drug in vivo, especially significantly improved the absorption of the drug under the condition of empty stomach, and significantly reduced the food effect.

Specific embodiments according to the present disclosure are provided as above, which are not any limitation to the scope there of. All equivalents of the present disclosure, or direct or indirect applications in other relevant technical fields are equally included within the scope of patent protection of the present disclosure.

1. A pharmaceutical composition, comprising Darolutamide or a pharmaceutically acceptable salt thereof as active ingredient, a carrier material, and a formulation modifying agent; wherein
the formulation modifying agent is one or more selected from the group consisting of: sodium lauryl sulfate, vitamin E polyethylene glycol succinate, Poloxamer, polyoxyethylene hydrogenated castor oil, octadecanol, dibutyl sebacate, triethyl citrate, butyl citrate, glycerol, polyethylene glycol, lecithin, dioctyl sulfosuccinate sodium, sodium taurocholate, polysorbitol, polyoxyethylene alkyl ether, polyoxyethylene fatty acid ester, and polyoxyethylene castor oil; the carrier material is one or more selected from the group consisting of: hydroxypropylmethyl cellulose phthalate, hydroxypropyl methylcellulose, polyvinyl alcohol, polyvinyl pyrrolidone, hydroxypropyl methyl cellulose acetate succinate, polyvinyl caprolactam-polyvinyl acetate-polyethylene glycol graft copolymer, Eudragit, copovidone, methyl cellulose, ethyl cellulose, sodium carboxymethyl cellulose, hydroxypropyl cellulose, polyvinyl acetate, cyclodextrin, sodium carboxymethyl ethyl cellulose polyethylene oxide, cellulose acetate phthalate, and cellulose acetate trimellitate.

2. The pharmaceutical composition according to claim 1, wherein the formulation modifying agent is one or more selected from the group consisting of: sodium lauryl sulfate, vitamin E polyethylene glycol succinate, and triethyl citrate.

3. The pharmaceutical composition according to claim 1, wherein the carrier material is one or more selected from the group consisting of: hydroxypropylmethyl cellulose phthalate, hydroxypropyl methylcellulose, polyvinyl pyrrolidone, hydroxypropyl methyl cellulose acetate succinate, polyvinyl caprolactam-polyvinyl acetate-polyethylene glycol graft copolymer, and Eudragit.

4. The pharmaceutical composition according to claim 1, wherein
the pharmaceutical composition further comprises one or more selected from the group consisting of: a buffer, an acidifier, a stabilizer, and a preservative.

5. The pharmaceutical composition according to claim 1, wherein
the weight ratio of Darolutamide or the pharmaceutically acceptable salt as active ingredient to the carrier material is selected from the group consisting of about 1:0.5-1:8, about 1:1-1:4, and about 1:2-1:4.

6. The pharmaceutical composition according to claim 1, wherein
the pharmaceutical composition has a bulk density selected from the group consisting of about 0.40-0.50 g/mL, about 0.42-0.49 g/mL, and about 0.436-0.473 g/mL.

7. A solid formulation, comprising the pharmaceutical composition according to claim 1.

8. A process for preparing the pharmaceutical composition according to claim 1, comprising the steps of
(1) weighing each of the ingredients, and
(2) mixing the ingredients in step (1) to obtain a mixture and subjecting the mixture to hot-melt extrusion, to obtain the pharmaceutical composition.

9. A process for preparing the solid formulation according to claim 7, comprising the steps of
(1) weighing each of the ingredients,
(2) mixing the ingredients in step (1) to obtain a mixture and subjecting the mixture to hot-melt extrusion, to obtain the pharmaceutical composition,
(23) pulverizing the pharmaceutical composition of step (2) to obtain pharmaceutical composition granules,
(4) mixing the pharmaceutical composition granules of step (3) with a lubricant and other formulation modifying agent to obtain total mixed granules,
(5) pressing the total mixed granules to obtain the solid formulation; optionally, the process further comprises a step of:
(6) coating the solid formulation of step (5) to obtain a tablet.

10. A method for preventing or treating prostate cancer, comprising administering the pharmaceutical composition according to claim 1 to a subject in need thereof.

11. The pharmaceutical composition according to claim 4, wherein
the buffer is one or more selected from the group consisting of: citric acid buffer, malate buffer, maleate buffer, and tartrate buffer; and/or
the acidifier is one or more selected from the group consisting of: tartaric acid, carbonic acid, acetic acid, oxalic acid, and nitrous acid; and/or
the stabilizer is one or more selected from the group consisting of: methionine, lysine, and histidine;
the preservative is one or more selected from the group consisting of: benzyl alcohol, benzyl benzoate, methyl 4-hydroxybenzoate, propyl 4-hydroxybenzoate, vitamin E, and vitamin A palmitate.

12. The pharmaceutical composition according to claim 1, wherein
the weight ratio of Darolutamide or the pharmaceutically acceptable salt as active ingredient to the formulation modifying agent is selected from the group consisting of about 1:0.05-1:0.8, and about 1:0.1-1:0.5.

13. The pharmaceutical composition according to claim 1, wherein
the pharmaceutical composition has an angle of repose selected from the group consisting of about 20°-32°, about 21°-30°, and about 22°-28°.

14. A method for preventing or treating prostate cancer, comprising administering the solid formulation according to claim 7 to a subject in need thereof.
